(19) United States
(12) Patent Application Publication
(10) Pub. No.: US 2003/0129630 A1
(43) Pub. Date: Jul. 10, 2003

(54) Genetic markers associated with desirable and undesirable traits in horses, methods of identifying and using such markers

(75) Inventors: Girish N. Aakalu, Los Angeles, CA (US); Carlo J. Quinonez, Pasadena, CA (US); Daniel K. Meulemans, Pasadena, CA (US)

Correspondence Address: HOGAN & HARTSON L.L.P., 500 S. GRAND AVENUE, SUITE 1900, LOS ANGELES, CA, 90071-2611, US

(73) Assignee: Equigene Research Inc.

(21) Appl. No.: 10/273,307

(22) Filed: Oct. 17, 2002

Related U.S. Application Data
(60) Provisional application No. 60/332,572, filed on Nov. 21, 2001.
(60) Provisional application No. 60/330,249, filed on Oct. 17, 2001.
(60) Provisional application No. 60/330,181, filed on Oct. 17, 2001.
(60) Provisional application No. 60/330,182, filed on Oct. 17, 2001.

(51) Int. Cl. C12Q 1/68

(52) U.S. Cl. 435/6

(57) ABSTRACT

A method is disclosed for identifying genetic markers associated with desirable and undesirable traits in horses, including athletic performance, physical structure, injury susceptibility, and disease susceptibility. The method involves partial sequencing of the horse genome, polymorphism identification, and whole-genome linkage analysis. When identified, these markers are utilized to create assays for inherited predisposition of a horse toward important physical traits and disease. The present invention also relates to a method of predicting desirable and undesirable traits in horses utilizing genetic markers of the present invention.

This application claims the benefit of U.S. Provisional Application No. 60/332,572, filed Nov. 21, 2001, U.S. Provisional Application No. 60/330,249, filed Oct. 17, 2001, U.S. Provisional Application No. 60/330,181, filed Oct. 17, 2001, and U.S. Provisional Application No. 60/330,182, filed Oct. 17, 2001.

### BACKGROUND OF THE INVENTION

The present invention relates to genetic markers associated with various desirable and undesirable traits in horses, particularly in thoroughbred horses, including athletic performance, physical structure, injury susceptibility, and disease susceptibility. The present invention also relates to methods for identifying such genetic markers and methods of their use in the prediction of horse performance as well as in the study of human athletic performance and disease susceptibility.

Description of the Prior Art

Currently, very little is known about the genetics of athletic performance and disease in horses. Presently, horses can be screened only for two genetic disorders, hyperkalaemic periodic paralysis (HYPP) and severe combined immunodeficiency disease (SCID).

HYPP is a genetic disorder effecting quarter horses that results in muscle spasms and paralysis (Rudolph, J., Spier, S. et al. (1992), “Periodic paralysis in quarter horses—a sodium-channel mutation disseminated by selective breeding,” Nature Genetics 2(2): 144-147; Shin, E., L. Perryman, et al. (1997), “Evaluation of a test for identification of Arabian horses heterozygous for the severe combined immunodeficiency trait,” J. American Veterinary Medical Association 211(10): 1268). A PCR-based genetic test is available to identify horses with the HYPP disease allele. Breeders use this information to minimize the prevalence of HYPP in their stock or to identify animals needing treatment.

SCID is a genetic disease of the immune system effecting Arabian horses (Don-van't Slot, H. and J. van der Kolk (2000), “Severe-Combined-Immunodeficiency-Disease (SCID) in the Arabian horse: a review.” Tijdschrift Voor Diergeneeskunde 125(19): 577-581). Horses carrying the SCID disease allele have dysfunctional immune systems. As with HYPP, a genetic test is available that identifies carriers of the defective SCID gene.

Both the horse HYPP and SCID genes were uncovered by a candidate gene approach. Researchers observed that similar genetic disorders affect human patients. Previous genetic linkage studies in humans identified the loci responsible for the human diseases. This information was successfully used to create diagnostic assays for horse HYPP and SCID. While testing for these two genetic markers is important for some horses, neither marker is used for thoroughbred horses. There are no genetic screens for diseases in thoroughbreds, though some microsatellite (Cho, G., B. Kim, et al. (2000), “Usefulness of microsatellite markers for horse parentage testing,” Korean Journal Of Genetics 22(4): 281-287) and restriction fragment length polymorphism (RFLP) based genetic tests are available to determine parentage.

Commercial breeding consultants also trace pedigrees to determine if a genetic predisposition towards greater heart size is present in a horse's lineage. It is believed that a gene referred to as an X-factor may be responsible for this performance-enhancing trait. The exact location and identity of the X-factor is unknown, although pedigree analyses suggest that it is located on the X-chromosome (Haun, Marianna, (1996), “The X Factor: what it is and how to find it: the relationship between heart size and racing performance,” The Russell Meerdink Company Ltd., Neenah Wis.). However, such pedigree analysis is limited in its predictive ability and does not have a molecular basis.

To date, the most sophisticated effort to characterize the horse genome has been made by a small collaboration of labs called the Horse Genome Project. A major goal of the Horse Genome Project is to identify genes associated with various diseases via genome-wide linkage studies. To achieve this goal, Horse Genome Project researchers are slowly identifying microsatellite markers in the horse genome. Using conventional laboratory methods, the horse genome project has identified and mapped 400 genetic markers in six years (Swinbume, J., C. Gerstenberg, et al. (2000), “First comprehensive low-density horse linkage map based on two 3-generation, full-sibling, cross-bred horse reference families.” Genomics 66(2): 123-134). However, this rough map has not been used in linkage studies to identify markers for positive or negative traits in horses.

In recent years, horse synteny maps have also been generated by a variety of methods (Caetano, A., L. Lyons, et al. (1999), “Equine synteny mapping of comparative anchor tagged sequences (CATS) from human Chromosome 5,” Mammalian Genome 10(11): 1082-1084.; Shiue, Y., L. Bickel, et al. (1999), “A synteny map of the horse genome comprised of 240 microsatellite and RAPD markers,” Animal Genetics 30(1): 1-9). These synteny maps identify large regions of homology between genomes of different species and aid in searches for horse homologs of human disease genes. However, the synteny maps have not been utilized to find new disease genes in horses.

Currently, horse bloodstock breeders must rely on biomechanical, geometric, and physiological criteria to evaluate young adult horses (14 months and older) for their inherited racing and breeding potential. The size and relative positions of major muscles in the fore and hind limbs are measured to estimate stride power. Slow-motion videography is utilized to evaluate the efficiency of a horse's gait. Blood pressure and ultrasound are used to determine heart size, thickness, and stroke volume. However, because a phenotype of an adult horse depends on the interaction of its genotype and environment, an adult phenotype does not provide an accurate prediction of the horse's genetic potential. In addition, parental phenotype is a poor predictor of offspring genotype. Phenotypically superior horses often produce below average foals, demonstrating the limitations of phenotypic analysis in predicting breeding potential.

### SUMMARY OF THE INVENTION

In view of the above-noted shortcomings of conventional genetic screening methods and because of the economic importance of thoroughbred horses to the horse racing industry, it is an object of the present invention to provide genetic markers associated with various desirable and undesirable traits in horses, including performance and susceptibility to diseases. It is another object of the present invention to provide methods for identifying such genetic markers. Also, it is an object of the present invention to provide methods of using such genetic markers and genes alone or in combination with the more traditional phenotypic analyses (e.g., biomechanical, geometric and physiological analysis), in the prediction of horse performance and predisposition towards physical traits and diseases as well as in the study of human athletic performance and disease susceptibility. It is a further object of this invention to develop a test that utilizes genetic information to predict athletic performance, disease susceptibility, racing, or breeding potential of a horse, and to develop appropriate training programs for the horse based on its genetic predisposition to desirable and undesirable traits.

To achieve these and other objectives, the present invention provides a method for uncovering genetic markers in horses. The method comprises (a) identifying a plurality of polymorphic markers within a population of horses; (b) determining genotypes of at least some horses in the population for at least some of the plurality of polymorphic markers; (c) determining at least one phenotype of at least some horses in the population; (d) comparing the determined genotypes to at least one determined phenotype; and (e) determining polymorphic markers that are statistically correlated to the phenotype.

In another aspect, the present invention provides genetic markers identified by the above-described method. In one embodiment, the genetic markers are associated with desirable and undesirable traits in horses, including athletic performance, physical structure, lung capacity, and injury and disease susceptibility.

The identified markers may be used to create assays to determine a horse's predisposition towards certain physical traits and diseases. The identified markers also may be used to discover human genes responsible for similar traits in humans and other animals. Accordingly, the invention also provides methods of using markers identified by the above-described method to select horses with the desired traits for training at a young age. The invention also provides methods for the prediction of the appropriate training regime for a particular horse, for example, based on its injury susceptibility, as determined using the genetic markers of the present invention.

The invention constitutes a dramatic improvement over current methods of finding genetic markers for athletic performance, physical structure, injury susceptibility, and diseases in horses. This method is novel in its use of partial genome sequencing, polymorphism searches, and genome-wide linkage analysis to find markers for specific traits in horses, including athletic performance, physical structure, injury susceptibility, and diseases. Prior to the present invention, these techniques have not been applied to the field of horse genetics. Additionally, experts in the field have dismissed genome-wide linkage scans for athletic performance genes in horses as impractical.

Additionally, the methods of the present invention surpass the Horse Genome Project's microsatellite-based strategy in speed, convenience, and resolution. The process of finding useful microsatellites is labor intensive, especially in a highly inbred strain such as thoroughbreds (Tozaki, T., S. Mashima, et al. (2001), “Characterization of equine microsatellites and microsatellite-linked repetitive elements (eMLREs) by efficient cloning and genotyping methods,” DNA Research 8(1): 33-45). The present method is based on identification of polymorphic markers, such as a single nucleotide polymorphisms (SNP), by high-throughput sequencing technology, which allows for the generation of higher resolution marker maps much faster than conventional microsatellite screens.

Also, the present method is superior to the candidate gene method, which relies upon human genetic linkage studies to identify important genes. This is because only a subset of traits is tractable to this kind of analysis in humans. Complex traits such as athletic ability and physical structure are very difficult to study in humans because of the environmental and genetic variability inherent in human populations (Terwilliger, J. and K. Weiss (1998), “Linkage disequilibrium mapping of complex disease: fantasy or reality?” Current Opinion in Biotechnology 9(6): 578-594).

The genetic markers and genes of the present invention can be advantageously used either alone or in combination with more traditional phenotypic analyses (e.g., biomechanical, geometric and physiological analysis) to predict horse performance and provide improved bloodstock consultation, including recommendations on utilization of the genetic potential of tested horses. It is believed that the present method will be particularly advantageous when applied to thoroughbred horses, where the degree of environmental and genetic variability is greatly reduced. The methods of the invention also provide knowledge that can be used in the study of human athletic ability and injury susceptibility.

It is to be understood that both the foregoing general description and the following detailed description are exemplary and explanatory and are intended to provide further explanation of the invention as described and claimed.

### BRIEF DESCRIPTION OF THE FIGURES

The above-mentioned and other features of the present invention and the manner of obtaining them will become more apparent, and will be best understood, by reference to the following description, taken in conjunction with the accompanying drawings, in which:

FIG. 1 outlines the process of developing a database of SNPs linked to important traits in horses, in accordance with one embodiment of the present invention.

### DETAILED DESCRIPTION OF THE EMBODIMENTS OF THE INVENTION

The present invention provides a method for identifying genetic markers in horses. The method comprises (a) identifying a plurality of polymorphic markers within a population of horses; (b) determining genotypes of at least some horses in said population for at least some of said plurality of polymorphic markers; (c) determining at least one phenotype of at least some horses in said population; (d) comparing the determined genotypes to at least one determined phenotype; and (e) determining polymorphic markers that are statistically correlated to said at least one phenotype. In one embodiment, the genetic markers are associated with athletic performance, physical structure, injury susceptibility, and disease susceptibility in thoroughbred horses.

Identification of Markers

Initial identification of polymorphic marker loci is accomplished by partial sequencing of individual or pooled thoroughbred genomic DNA and a subsequent search for single nucleotide polymorphisms (SNPs) and insertions or deletions (Indels). For the purposes of the present invention, SNPs are DNA sequence variations between individual horses that occur when a single nucleotide (A, T, C, or G) in the genome sequence is changed. For the purposes of the present invention, Indel is a gain (insertion) or loss (deletion) of one or more nucleotides at a specific position in DNA sequences obtained from different horses. For the purposes of the present invention, a polymorphic marker may comprise an SNP or Indel.

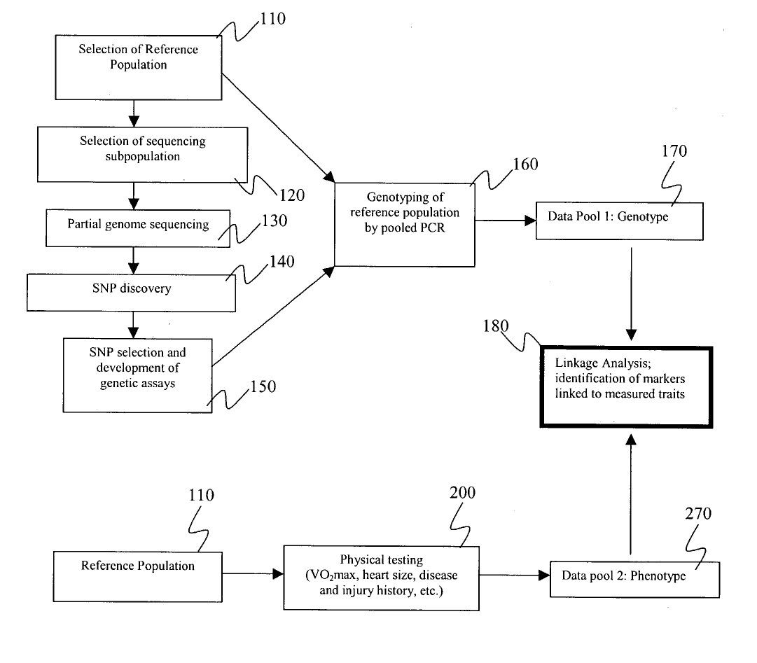

In one embodiment depicted in FIG. 1, the plurality of single nucleotide polymorphisms is identified as follows. A reference population of horses 110 is selected. A subset of horses 120 is chosen from the reference population. The DNA obtained from the horses in the subset is partially sequenced 130, either separately for each horse or pooled. Polymorphic markers differing among the horses are identified 140 through comparison of the sequences obtained from different horses. When pooled DNA is used, polymorphic markers are identified by noting polymorphisms within the pooled sequence data.

In one embodiment of the present invention, the reference population 110 comprises at least more than about 30 horses, more preferably at least 50, and even more preferably at least 100 horses. In another embodiment, the reference population comprises at least 300 horses. The number of the horses selected can be determined by one of skill in the art depending on the amount of pedigree information available for the reference population.

Although any horses may be used for the purposes of the present invention, in one embodiment, the horses are thoroughbred horses. Although any subset of the horse reference population may be selected for identification of polymorphic markers, in one embodiment, about 10% of the horses in the population are selected for the subset. For instance, in one embodiment that is discussed in Example 1, a subset of 25 thoroughbred horses out of a population of 276 thoroughbred horses was selected for identification of polymorphic markers.

In one embodiment, as illustrated in Example 1, genomic DNA is extracted from each of the horses in the subset and pooled to give a pooled subset. The pooled genomic DNA is digested with a restriction enzyme and the digested DNA is separated on an agarose gel. A band corresponding to DNA fragments of a predetermined size is cut from the gel and the DNA is extracted from the agarose. The pooled DNA fragments are subcloned into a plasmid and introduced into E. coli by electroporation. Clones are grown on agar, and an automated colony-picking machine, such as Q-Bot made by Genetix, Inc. (New Milton, UK), is used to select clones, from which DNA is extracted.

Although DNA bands of any size may be used for identification of polymorphic markers, in one embodiment a band corresponding to DNA fragments of about 500-600 base pairs was chosen because this band size corresponds to high quality sequence in the average sequencing run. Fragments larger than 600 bp may have low-quality sequence toward the end of the sequencing run and fragments smaller than 500 may have progressively less chance of containing an SNP or an Indel, Although any number of clones can be selected, typically, at least 10,000 clones are selected. Preferably, at least 15,000 clones are selected. Most preferably, at least 18,000 clones are selected. For example, in one embodiment 20,000 clones are selected.

Plasmids derived from the various selected clones are sequenced using a fluorescent capillary electrophoresis DNA sequencing system, such as PRISM™ 3706 DNA Sequencer available from Applied Biosystems (Foster City, Calif.). The sequence is analyzed according to the method of Altschuler et al. (Nature (2000) 407:513-516), which is incorporated herein by the reference, to determine the presence of polymorphic markers, such as SNPs and Indels, in the analyzed sequences using the neighborhood quality standard (NQS) method. Typically, at least 500 polymorphic markers are identified. Prefereably, at least 750 polymorphic markers are identified. Most preferably, at least 1000 polymorphic markers are identified. For example, in one embodiment between 1000 and 2000 SNPs are identified. This process can be scaled up to find more SNPs by using a plurality of restriction enzymes to increase the number of non-identical fragments in the 500-600 bp range. Typically, for each additional restriction enzyme used the numbers of clones selected and SNPs identified will double.

Determining Genotypes

All or a selection of the polymorphic markers that are identified 150 may be chosen to determine genotypes of the horses in the reference population 110. The horse genotypes are preferably determined at about 500 to about 30,000 polymorphic marker loci. In one embodiment, a subset of 1000-2000 polymorphic markers is chosen based upon the degree of polymorphism and genomic location of the various markers. Preferably, the polymorphic markers are selected to give an approximately evenly spaced coverage of the genome.

Genotypes can be determined by a large number of techniques that allow for the detection of the particular genetic marker, including for example, methods for detecting SNPs and Indels. Some methods for determining genotypes have been reviewed recently (Pui-Yan Kwok, (2001) Methods For Genotyping Single Nucleotide Polymorphisms, Annu. Rev. Genomics Hum. Genet., 2:235-58; Kirk, B. W. et al. (2002), Single Nucleotide polymorphism seeking long term association with complex disease, Nucleic Acids Research 30: 3295-3311.) Such techniques include, but are not limited to, detection on microarrays with fluorescent detection; molecular beacon genotyping; 5′ nuclease assays; allele-specific polymerase chain reaction (PCR); allele-specific primer extension; arrayed primer extension; homogenous primer extension assays; primer extension with mass spectrometry detection; pyrosequening; multiplex primer extension; ligation with rolling circle amplification (RCAT); homogenous ligation; multiplex ligation; flap endonuclease assays, for example INVADER™ assays available from Third Wave Technologies (Madison, Wis.); mismatch scanning assays. One of skill in the art will be able to determine an appropriate technique for determining genotypes depending on the nature of the polymorphic markers (SNP versus Indel) and the number of markers being queried.

The present invention does not impose a restriction on selection of a technique for determining genotypes of horses at the identified polymorphic markers as long as the chosen technique provides an acceptable level of accuracy. In one embodiment, the technique chosen for determining the genotype can be performed with at least 90% accuracy, more preferably at least 95% accuracy and even more preferably at least 98% accuracy. For example, in one embodiment, genotyping of the population of horses at the polymorphic marker loci is accomplished by standard high-throughput PCR-based methods.

Referring again to FIG. 1, in one embodiment, the genotypes of the reference horse population is determined 160 at each of the selected polymorphic markers to result in a pool of data (Data Pool 1) 170. Data Pool 1 represents the genotype of each horse in the reference horse population at each selected polymorphic marker. When the polymorphic marker is a single nucleotide polymorphism, there are four possible entries for each polymorphism: A, G, C and T. The data of the Data Pool 1 may be represented in a simple two-dimensional matrix. For each horse or group of horses for which genotypes have been determined at the plurality of marker loci, a database entry will include a horse identifier entry and the genotype at each such locus. Such matrix may be stored and manipulated using a computer system known to those skilled in the art. For example, such computer system may have an input device, a memory, a processor and an output or display device.

Phenotype Analysis

A variety of phenotypes may be measured for each horse in the reference population, especially those related to traits of interest, including those related or thought to relate to performance characteristics, physical structure or disease susceptibility. These measurements may include, but are not be limited to, limb length, limb angle, muscle volume, resting heart rate, time to resting heart rate after physical exertion, blood pressure, maximum oxygen uptake (VO₂max), maximum carbon dioxide production (VCO₂max), blood volume at rest and exercise, rebreathing measurements of lung volumes, maximum sprint speed, heart size, history of joint, skin, and cardiovascular disease, orthopaedic diseases, chronic obstructive pulmonary disease, pulmonary “bleeding” during extreme exertion, muscle diseases like exertional rhabdomyolysis, immune system disorders causing sarcoid tumors, and insect bite hypersensitivity.

Variables chosen for phenotypic determination may have a numerical format or can be grouped into ranges to form categorical variables. For example, a continuous variable such as a horse's maximum sprint speed can be grouped into several categories, such as fastest horses, having a sprint speed of over 17.5 meters/second; fast horses, having a sprint speed of between about 16 and 17.5 meters/second, average horses having a sprint speed of between 15 and 16 meters/second. As will be apparent to one of skill in the art of statistical analysis, the segmentation of such variables can be chosen through groups of categorical variables according to the distribution of the continuous variable.

Referring to FIG. 1, in one embodiment, the phenotype is determined 200 of each of the horses in the reference population. Each phenotype is stored as a record in a database (Data Pool 2). Data Pool 2 includes a horse identifier entry and an entry for a value for each phenotype determined for the horse. The data may be stored on a computer system for a comparison with the first data pool (Data Pool 1).

Comparing Genotypes and Phenotypes

According to the methods of the invention, the first data pool having the genotype information for each of the horses and the second data pool having the phenotype information are compared to determine the polymorphic markers that are associated with desirable or undesirable traits, such as athletic performance, physical structure, injury susceptibility, and/or disease susceptibility. The comparison can be made through a computational analysis of the statistical correlations between the phenotypes and the genotypes. Such linkage analysis can be performed by methods known to one of skill in the art, including techniques described herein. In one such embodiment, a correlation matrix is generated comparing each phenotype and genotype.

The statistical comparison may further include pedigree information. The relationship of the various horses within the reference population can be used to perform affected sibling pair analyses or affected relative pair linkage analyses. In one embodiment, pedigree data is adapted to affected pedigree methods of linkage analysis exemplified by the software package GENEHUNTER™, Whitehead Institute, Cambridge, Mass. (Kruglyak L, Daly M, Reeve-Daly M, and Lander E., “Parametric and Nonparametric Linkage Analysis: A Unified Multipoint Approach,” American Journal of Human Genetics 58 (1996): 1347-1363), incorporated herein by the reference.

The comparison between the two data pools may be made using any one of a number of commercial genetic correlation programs, exemplified by the LINKAGE© package (Lathrop, Lalouel, Julier, Ott, Proc. Natl. Acad. Sci., 81, 3443-3446 (1984); Lathrop, Lalouel, Am. J. Hum. Genet, 36, 460-465 (1984); Lathrop, Lalouel, White, Genet. Epid., 3, 39-52 (1986); Young, Weeks, Lathrop, Am. J. Hum. Genet. Suppl., 57(4), A206 (1995)), incorporated herein by the reference.

This correlation may take the form of a bulk segregant analysis, whereby individual horses with similar phenotypes are grouped together and genotyped en masse using a pooled PCR approach. In this strategy, equal portions of DNA from each horse in a group are pooled and genotyped as a single sample at each marker locus. The allelic frequency of the phenotypic groups is then deduced according to the method of Germer (Germer, S., M. Holland, et al. (2000), “High-throughput SNP allele-frequency determination in pooled DNA samples by kinetic PCR,” Genome Research 10(2): 258-266.) genetic markers showing a strong correlation with any of the measured physical traits are identified.

Genetic Markers Associated with Desirable and Undesirable Traits in Horses

In another aspect, the present invention provides genetic markers identified by the above-described method. In one embodiment, the genetic markers are associated with desirable and undesirable traits in horses, including athletic performance, physical structure, lung capacity, and injury and disease susceptibility. The resulting database of genetic markers may be used as a basis for diagnostic genetic assays for horses and a starting point for the identification of genes involved with the measured phenotypes. The DNA sequence of alleles at a locus may be used to design PCR primers for rapid genotyping of individual horses. This genotyping may be used as an assay for a horse's genetic predisposition towards desirable or undesirable traits, including athletic potential, physical structure (size of the heart and lungs, limb length, limb angle, muscle volume, etc.) and disease susceptibility. The DNA sequences of markers may also be used to isolate DNA surrounding the marker and map the marker using the human genome sequence as a reference. Localization of the marker in the horse genome will allow discovery of genes associated with the phenotypes observed and facilitate basic research into the function of these genes.

Predicting Undesirable and Desirable Traits in Horses

The invention also includes a method for predicting desirable or undesirable traits in horses. This method is believed to have a particular value in thoroughbred bloodstock consultation. According to the method, the genotype of a horse determined at one or more polymorphic markers to assess the genetic potential of the horse. More specifically, the genotype is determined at polymorphic markers that relate to the desirable and/or undesirable traits in horses, including disease susceptibility, physical structure, and athletic performance. According to the methods of the invention, the genotype analysis for a given horse will allow for the prediction of a probability for the horse to have certain traits. Such information can be used to counsel a horse owner or other interested parties.

The genotype of a horse may be determined by any of the techniques listed above or any other techniques known to one of skill in the art. DNA may be extracted from a horse tissue, including for example, plucked hair follicles and blood samples. The genotype can then be determined, for example using a PCR assay with allele-specific primers. The presence of a given allele is determined by the quantity of the resulting reaction product. By determining the genotype of horses at selected loci, their genetic predispositions towards performance, injury, and disease may be assessed. Breeders may be advised as to which of their young horses are most suited for racing and which pairs of horses are the most genetically compatible (i.e. will produce superior offspring). Trainers may be advised as to training regimens for each horse. According to the methods of the invention, for example, an owner of a horse with a high susceptibility to joint diseases may be advised to train the horse less aggressively than a horse lacking such a susceptibility.

Transfer of Horse Genetic Data to Humans

After finding the markers strongly linked to the traits of interest, homologous human loci can be identified. Computer searches of published human DNA sequence with the horse sequence surrounding the marker will suggest in which large human genomic region the associated genes will be found. For example, in one embodiment, the partial sequence runs of about 500-600 nucleotides are used to identify bacterial artificial chromosome (BAC) clones from a horse genome library that contain DNA having the polymorphic marker associated with a given gene. These BAC clones are sequenced at adjacent regions to give a longer piece of sequence information that may be used to make a comparison with human genomic DNA sequences. In one embodiment, the sequence comparison is made with a simple software method such as those embodied in the BLAST programs (Altschul, S. F., Gish, W., Miller, W., Myers, E. W. & Lipman, D. J., (1990) “Basic local alignment search tool,” J. Mol. Biol. 215:403-410; Gish, W. & States, D. J., (1993) “Identification of protein coding regions by database similarity search,” Nature Genet. 3:266-272; Madden, T. L., Tatusov, R. L. & Zhang, J. (1996) “Applications of network BLAST server” Meth. Enzymol. 266:131-141; Altschul, S. F., Madden, T. L., Schaffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402; Zhang, J. & Madden, T. L. (1997) “PowerBLAST: A new network BLAST application for interactive or automated sequence analysis and annotation.” Genome Res. 7:649-656). The identified region of the human genome will allow for the identification of candidate genes within the region that may be responsible for the trait linked with a polymorphic marker.

In another embodiment, the partial sequence runs of 500-600 nuleotides are directly used to search the human genome, without first identifying a horse BAC clone. In yet another embodiment, the partial sequence runs of 500-600 nucleotides are used to search a publicly available horse genome map, and the corresponding region of the human genome is found using a human/horse synteny map.

Utilization of the Pool of Human Genes

When derived by the methods of the present invention, the pool of human genes will represent genes with a high likelihood of being associated with athletic performance, injury, and disease susceptibility. Then, researchers may use this pool to find positive or negative acting alleles, and to develop diagnostic tests for these alleles. The set of genes may also be used directly as drug targets and may form a valuable resource for researchers investigating the genetic bases of athletic ability, injury and skeletomuscular disease susceptibility.

The foregoing is meant to illustrate, but not to limit, the scope of the invention. Indeed, those of ordinary skill in the art can readily envision and produce further embodiments, based on the teachings herein, without undue experimentation.

### EXAMPLE 1

A population of 276 thoroughbred horses is analyzed for the following phenotypes: maximum sprint speed; upper leg length; lower leg length; height; upper leg-lower leg angle; lung volume, maximal O2 uptake, red blood cell count, history of joint disease, orthopaedic diseases, chronic obstructive pulmonary disease, pulmonary bleeding during extreme exertion, exertional rhabdomyolysis, sarcoid tumors, and insect bite hypersensitivity. A subset of 25 of the 276 thoroughbred horses is selected as a sequencing subpopulation. Genomic DNA is then extracted from each of the 25 horses in the subset and pooled to give a pooled subset. The pooled genomic DNA is digested with the restriction enzyme BglII and the digested DNA is separated on an agarose gel. A band corresponding to DNA fragments of a size of about 500-600 base pairs is cut from the gel and the DNA is extracted from the agarose.

The pooled DNA fragments are subcloned into the plasmid M13mp19RFIDNA (Pharmacia, Peapack N.J.), introduced into E. coli by electroporation, and grown on agar according to standard methods (Sambrook J. and Russell D. W., 2001 Molecular Cloning a Laboratory Manual, Third Edition, Cold Spring Harbor Laboratory Press, Cold Spring Harbor, N.Y.). An automated colony-picking machine (Q-Bot, Genetix, Inc., New Milton, UK) is used to select 25,000 clones, from which DNA is extracted. The 25,000 plasmids derived from the various clones are sequenced using a fluorescent capillary electrophoresis DNA sequencing system (PRISM 3700 DNA Sequencer, Applied Biosystems, Foster City, Calif.). The sequence is analyzed according to the method of Altschuler et al. (2000) Nature 407:513-516) to determine the presence of SNPs in the analyzed sequences using the neighborhood quality standard (NQS) method. About 1,721 SNPs are identified in the pool.

All 276 horses in the reference population are genotyped at each of the 1,721 SNPs using an extension-based approach using a fiber optic microarray (ILLUMINA BEADARRAY, Illumina, San Diego, Calif.) having each of the 1,721 SNPs represented. The genotype data is recorded in a database for each horse at each readable genotype. The genotype database and phenotype database are analyzed using the LINKAGE© software package.

The present invention may be embodied in other specific forms without departing from its essential characteristics. The described embodiment is to be considered in all respects only as illustrative and not as restrictive. The scope of the invention is, therefore, indicated by the appended claims rather than by the foregoing description. All changes which come within the meaning and range of the equivalence of the claims are to be embraced within their scope.

What is claimed is:

1. A method for identification of genetic markers in horses comprising:
(a) identifying a plurality of polymorphic markers within a population of horses;
(b) determining genotypes of at least some horses in said population for at least some of said plurality of polymorphic markers;
(c) determining at least one phenotype of at least some horses in said population;
(d) comparing the determined genotypes to at least one determined phenotype; and
(e) determining polymorphic markers that are statistically correlated to said at least one phenotype.

2. The method of claim 1, wherein the population of horses comprises at least 30 horses.

3. The method of claim 2, wherein the population of horses comprises at least 300 horses.

4. The method of claim 1, wherein the polymorphic marker comprises a single nucleotide polymorphism, an insertion or a deletion.

5. The method of claim 1, wherein step (a) further comprises:
(f) isolating a genomic DNA sample from a subset of the population;
(g) partially sequencing the genomic DNA; and
(h) comparing DNA sequences to identify the presence of polymorphic markers in the sequence.

6. The method of claim 5, wherein the genomic DNA is sequenced separately for each horse in the subset.

7. The method of claim 5, wherein the genomic DNA from at least some horses in the subset is pooled prior to sequencing.

8. The method of claim 5, wherein step (g) further comprises:
(i) fragmenting the DNA to provide a plurality of DNA fragments; and
(j) determining a plurality of nucleotide sequences of a number of the plurality of DNA fragments.

9. The method of claim 8, wherein the fragmenting step comprises digesting DNA with a restriction endonuclease.

10. The method of claim 5, wherein step (h) is carried out using the neighborhood quality standard method.

11. The method of claim 1, wherein at least 500 polymorphic markers are identified.

12. The method of claim 1, wherein horse genotypes are-determined for all identified polymorphic markers.

13. The method of claim 12, wherein horse genotypes are determined for a subset of the identified polymorphic markers comprising at least 500 polymorphic markers.

14. The method of claim 13, wherein the subset of the identified polymorphic markers is selected to give an approximately evenly spaced coverage of the horse genome.

15. The method of claim 1, wherein step (b) of determining genotypes comprises a technique selected from the group consisting of detection on microarrays with fluorescent detection; molecular beacon genotyping; 5′ nuclease assays; allele-specific polymerase chain reaction (PCR); allele-specific primer extension; arrayed primer extension; homogenous primer extension assays; primer extension with mass spectrometry detection; pyrosequening; multiplex primer extension; ligtion with rolling circle amplification (RCAT); homogenous ligation; multiplex ligation; flap endonuclease assays; and mismatch scanning assays.

16. The method of claim 15, wherein the technique is selected based on a type of polymorphic marker used and a number of polymorphic markers being queried.

17. The method of claim 1, wherein the phenotype measured is selected from the group consisting of limb length, limb angle, muscle volume, resting heart rate, time to resting heart rate after physical exertion, blood pressure, maximum oxygen uptake, maximum carbon dioxide production, blood volume at rest and exercise, rebreathing measurements of lung volumes, maximum sprint speed, heart size, history of joint, skin, and cardiovascular disease, orthopaedic diseases, chronic obstructive pulmonary disease, pulmonary “bleeding” during extreme exertion, muscle diseases like exertional rhabdomyolysis, immune system disorders causing sarcoid tumors, and insect bite hypersensitivity.

18. The method of claim 1, wherein comparing step (d) comprises statistical correlation of the determined genotypes and phenotypes.

19. The method of claim 18, wherein comparing step (d) further includes a pedigree information.

20. A horse genetic marker identified by the method of claim 1.

21. A method for predicting desirable or undesirable traits in a horse comprising:
(a) identifying a plurality of polymorphic markers within a population of horses;
(b) determining genotypes of at least some horses in said population for at least some of said plurality of polymorphic markers;
(c) determining at least one phenotype associated with desirable or undesirable traits of at least some horses in said population;
(d) comparing the determined genotypes to at least one determined phenotype;
(e) determining polymorphic markers that are statistically correlated to said desirable or undesirable traits; and
(g) determining the genotype of the horse at one or more polymorphic markers linked to the desired or undesired traits.

22. The method of claim 21, wherein step (g) further comprises obtaining a DNA sample from the horse for determining the genotype of the horse.

23. The method of claim 22, wherein the DNA sample is extracted from a horse tissue or blood samples.

24. The method of claim 21, further comprising the step of determining the genetic predisposition of the horse to the desirable and undesirable traits based on the genotype of the horse at one or more polymorphic markers linked to the desired or undesired traits.

25. The method of claim 24, wherein the desired and undesired traits are selected from a group consisting of athletic performance, physical structure, and disease susceptibility.

26. The method of claim 25, further comprising the step of selecting horses suitable for racing based on their genetic predisposition toward athletic performance.

27. A method for identification of human genes associated with desirable or undesirable traits comprising:
(a) identifying a plurality of polymorphic markers within a population of horses;
(b) determining genotypes of at least some horses in said population for at least some of said plurality of polymorphic markers;
(c) determining at least one phenotype associated with desirable or undesirable traits of at least some horses in said population;
(d) comparing the determined genotypes to at least one determined phenotype;
(e) determining polymorphic markers that are statistically correlated to said desirable or undesirable traits; and
(g) identifying human genes homologous to polymorphic markers linked to the desired or undesired traits.

28. The method of claim 27, wherein the desired and undesired traits are selected from a group consisting of athletic ability, injury susceptibility, and disease susceptibility.

29. A method of predicting injury and disease susceptibility in humans comprising:
(a) using method of claim 27 to identify human genes associated with the injury and disease susceptibility;
(b) determining positively and negatively acting alleles; and
(c) testing DNA of the patient for the positively and negatively acting alleles.

30. Human genes identified by the method of claim 27.
